(19) United States
(12) Patent Application Publication
(10) Pub. No.: US 2011/0124090 A1
(43) Pub. Date: May 26, 2011

(54) GENE INVOLVED IN THE BIOSYNTHESES OF LYCOPENE, RECOMBINANT VECTOR COMPRISING THE GENE, AND TRANSFORMED MICROORGANISM WITH THE RECOMBINANT VECTOR

(75) Inventors: Nahm Ryune Cho, Daejeon (KR); Min Soo Park, Daejeon (KR); Dong Hyun Lee, Daejeon (KR); Ho Seung Chung, Seoul (KR); Jong Keun Kim, Daejeon (KR)

(73) Assignees: SK ENERGY CO., LTD., SEOUL (KR); AMICOGEN CO., LTD., GYUNGSANG-NAM-DO (KR)

(21) Appl. No.: 12/594,659

(22) PCT Filed: Apr. 7, 2008
(86) PCT No.: PCT/KR2008/001960

§ 371 Date: Jan. 8, 2010

(30) Foreign Application Priority Data
Apr. 5, 2007 (KR) 10-2007-0033680

(51) Int. Cl. C12N 1/21; C07H 21/04; C12N 15/70

(52) U.S. Cl. 435/252.33; 536/23.2; 435/320.1

(57) ABSTRACT

There are provided genes involved in the biosynthesis of lycopene and having DNA sequences set forth in SEQ ID NO: 1, SEQ ID NO: 3 and SEQ ID NO: 5 encoding proteins required for the biosynthesis of lycopene, a recombinant vector comprising at least one of the genes, and a mi [symbol] croorganism transformed with the recombinant vector and having a high content of lycopene. The lycopene is obtained at a yield of 15.3 mg/L and a content of 4.2 mg/gDCW when the recombined E. coli with the crt genes is cultivated, and the lycopene is also obtained with the maximum content of 5.4 mg/gDCW when a microorganism is transformed with the combination of the gene of the present invention and the known genes. Therefore, provided is the lycopene-producing strain having a more increased content of lycopene per dry cell weight than the known lycopene-producing strain with the genes. Accordingly, the genes may be useful to mass-produce lycopene in microorganisms, and also to mass-produce carotenoids.

### TECHNICAL FIELD

The present invention relates to a gene involved in the biosynthesis of lycopene, a recombinant vector comprising the gene and a transformed microorganism with the recombinant vector, and more particularly, to a gene required for the biosynthesis of lycopene and having DNA sequences of SEQ ID NO: 1, SEQ ID NO: 3 and SEQ ID NO: 5, a recombinant vector comprising at least one gene selected from the group consisting of the genes, and a transformed microorganism with the recombinant vector.

### BACKGROUND ART

Lycopene is one of the carotenoid pigments. Carotenoid is a C40 isoprenoid compound having antioxidant activity, and belongs to a group of pigments having yellow, red and orange colors depending on their molecular structures. For example, the carotenoid includes β-Carotene, lycopene, lutein, astaxanthin, zeaxanthin, etc., and it has been used as a nutrient supplement, a medical supply, an edible coloring agent and an animal fodder additive.

Among them, the lycopene has a molecular structure represented by Formula I, and is a lipid-soluble substance that forms a molecular body of a red pigment in tomato, watermelon, grapes or the like, and has a very low polarity. Like other carotenoids, the lycopene has antioxidant and anticancer activities.

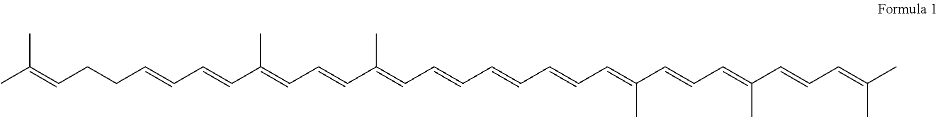

According to the researches that have been achieved up to now, a team led by Omer in the Karmanos Cancer Center in Detroit (U.S.) in the year 2000 reported that lycopene suppresses the metastasis of prostate cancer (Omer Kucuk et al., Cancer Epidemiology, 10, 861-869, 2001). Department of Allergy at Hasharon Hospital (Tel Aviv, Israel) and a lycopene manufacturer, LycoRed, confirmed that lycopene has an effect to relieve asthma symptoms in patients with exercises-induced asthma (I. Neuman et al., Allergy, 55, 1184-1189). Also, Department of Public Health at University of Kuopio reported clinical trial results that lycopene has superior protective effects on myocardial disease and ateriosclerosis (Tuna Rissanen et al., Exp Biol Med (Maywood), 227, 900-907, 2002).

An in vivo biosynthesis pathway of carotenoid is shown in FIG. 1.

Glycerol and glucose assimilated into living organisms are metabolized into isopentenyl pyrophosphate (hereinafter, referred to as ‘IPP’ or dimethylallyl pyrophosphate (hereinafter, referred to as ‘DMAPP’ when they are subject to a 2-C-methyl-D-erythritol-4-phosphate pathway (MEP pathway) or a mevalonate pathway (MVA pathway), and the IPP or the DMAPP is metabolized into farnesyl pyrophosphate (hereinafter, referred to as ‘FPP’ that is an important intermediate in the general isoprenoid pathway through several subsequent processes. The FPP and IPP is converted into geranylgeranyl pyrophosphate (hereinafter, referred to as ‘GGPP’ by geranylgeranyl pyrophosphate synthase encoded by crtE gene. Then, the GGPP is converted into phytoene by phytoene synthase encoded by crtB gene, and the phytoene is metabolized into lycopene by phytoene desaturase encoded by crtI gene. Then, the lycopene is converted into β-carotene by crtY gene, and the β-carotene is converted into zeaxanthin by β-carotene hydroxylase encoded by crtZ gene, and the zeaxanthin is converted into astaxanthin by β-carotene ketolase encoded by crtW gene. Also, the lycopene may be metabolized into lutein by crtL and crtR genes.

As described above, a mevalonate pathway and a non-mevalonate pathway have been known as the biosynthesis pathway of isopentenyl diphosphate (IPP) that is a common precursor of carotenoids. In this case, it was known that the mevalonate pathway is present in most eucaryotes (for example, Saccharomyces cerevisiae), cytoplasm in plant cells, some bacteria (for example, Streptococcus pneumoniae and Paracoccus zeaxanthinifaciens) and malaria cells. The non-mevalonate pathway is present in most bacteria (for example, Escherichia coli (E. coli)), and chromatophore (plastid) in plant cells. That is, the gram-negative (−) bacteria, E. coli, biosynthesizes IPP using only the non-mevalonate pathway. However, wild-type E. coli may not produce lycopene since the wild-type E. coli does not have genes involved in the biosynthesis of carotenoids including lycopene.

There have already been many attempts to produce carotenoids including lycopene by introducing a differently derived gene into a microorganism, such as wild-type E. coli, that does not produce lycopene. Roche Vitamins, Inc. prepared a transformant E. coli whose lycopene content is 0.5 mg/gDCW by transforming Flavobacterium sp. R1534-derived crtE, crtB and crtI genes (Luis Pasamontes et al., US20040058410, 2004), and Amoco Corporation prepared a yeast strain producing lycopene with a content of 0.1 mg/g (milligram/gram) DCW by using Erwinia herbicola-derived crtI gene (Rodney L. Ausich et al., U.S. Pat. No. 5,530,189, 1996). Misawa et al. prepared an E. coli strain producing lycopene with a content of 1.03 mg/g (milligram/gram) DCW, and a Saccharomyces cerevisiae sp. strain having a lycopene content of 0.11 mg/g (milligram/gram) DCW by using crtE, crtB and crtI gene derived from Erwinia species and Agrobacterium aurantiacum (Norihiko Misawa, Journal of Biotechnology, 59, 169-181, 1998). Kirin Beer Kabushiki Kaisha produced lycopene in a microorganism using Erwinia uredovora-derived crtE, crtB, crtI genes, and therefore obtained an E. coli strain with a lycopene content of 2.0 mg/g (milligram/gram) DCW (Norihiko Misawa, et al., U.S. Pat. No. 5,429,939, 1995).

However, since the content of lycopene is too low as described above in the research results, it is difficult to develop an effective production process. In order to solve the above problems, the present invention provides a novel gene capable of producing a transformant having a higher lycopene content than that of the known genes, a vector comprising the novel gene, and a transformed microorganism with the vector.

Accordingly, the present inventors have attempted to improve the productivity of lycopene, and found that a microorganism having a higher lycopene content can be prepared from microorganisms that does not produce lycopene by isolating crtE, crtB and crtI genes involved in the biosynthesis of lycopene from metagenome library of seawater, cloning the crtE, crtB and crtI genes, sequencing the genes, introducing the genes into a vector, and therefore the present invention was completed on the basis of the above-mentioned facts.

### DISCLOSURE OF INVENTION

### Technical Problem

An aspect of the present invention provides a gene encoding a protein that is required for the biosynthesis of lycopene.

Another aspect of the present invention provides a recombinant vector comprising the gene.

Still another aspect of the present invention provides a recombined microorganism having an increased content of lycopene by using the recombinant vector.

### Technical Solution

According to an aspect of the present invention, there is provided a crtE gene encoding geranylgeranyl pyrophosphate synthase and having a DNA sequence set forth in SEQ ID NO: 1.

According to another aspect of the present invention, there is provided a crtB gene encoding phytoene synthase and having a DNA sequence set forth in SEQ ID NO: 3.

According to still another aspect of the present invention, there is provided a crtI gene encoding phytoene desaturase and having a DNA sequence set forth in SEQ ID NO: 5.

According to still another aspect of the present invention, there is provided a recombinant vector comprising at least one gene selected from the group consisting of the crtE gene set forth in SEQ ID NO: 1, the crtB gene set forth in SEQ ID NO: 3, and the crtI gene set forth in SEQ ID NO: 5.

According to yet another aspect of the present invention, there is provided a transformed microorganism with the recombinant vector.

### ADVANTAGEOUS EFFECTS

As described above, three novel crtE, crtB, crtI genes encoding proteins required for the biosynthesis of lycopene were cloned from metagenome library of seawater in the present invention. Also, it was confirmed that lycopene may be produced in E. coli that does not produce lycopene by employing the crt genes, and recombinant strains that have a higher lycopene content than those as prepared in the conventional technologies may be prepared by using only the new crt genes or its combinations with known crt genes. Therefore, the crt genes according to the present invention may be useful to produce carotenoids such as lycopene, and also very useful to mass-produce carotenoids including lycopene) in microorganisms.

### BRIEF DESCRIPTION OF THE DRAWINGS

FIG. 1 is a diagram illustrating a biosynthesis process of lycopene.

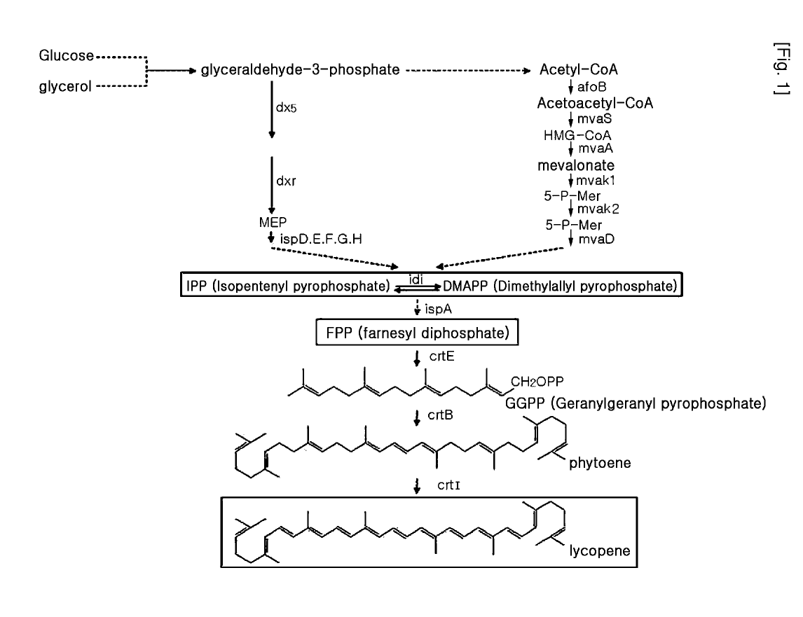

FIG. 2 is a diagram illustrating a cleavage map of a recombinant vector pT5-LYC-idi.

FIG. 3 is a diagram illustrating a cleavage map of a recombinant vector pT5-ErEBI.

FIG. 4 is a diagram illustrating a cleavage map of a recombinant vector pT5-ErBI.

FIG. 5 is a diagram illustrating a cleavage map of a recombinant vector pT-EF5.

FIG. 6 is a diagram illustrating a cleavage map of a recombinant vector pT-SF5.

FIG. 7 is a diagram illustrating a cleavage map of a recombinant vector pBF5-crt.

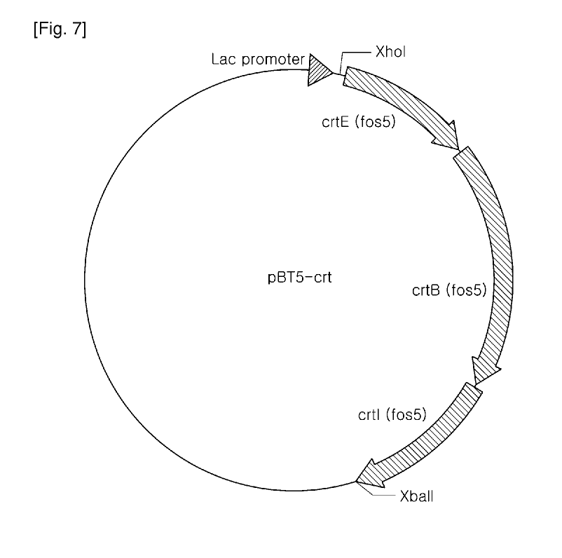

### BEST MODE FOR CARRYING OUT THE INVENTION

Hereinafter, exemplary embodiments of the present invention will be described in more detail with reference to the accompanying drawings.

In the present invention, crtE, crtB and crtI genes encoding proteins required for the biosynthesis of lycopene were cloned from metagenome library of seawater, a recombinant vector including these genes was constructed, and an E. coli strain that does not produce lycopene was transformed with the recombinant vector.

In addition, the present invention was completed by confirming that a content of lycopene is more increased by fermenting the transformed E. coli strain, when compared to those as prepared in the conventional researches.

According to the present invention, provided are genes encoding proteins required for the biosynthesis of lycopene and having DNA sequences set forth in SEQ ID NO: 1, SEQ ID NO: 3 and SEQ ID NO: 5, and the genes are obtained from a metagenome library of seawater. The DNA sequences of SEQ ID NO: 1, SEQ ID NO: 3 and SEQ ID NO: 5 encode amino acids (geranylgeranyl pyrophosphate synthase, phytoene synthase and phytoene desaturase) set forth in SEQ ID NO: 2, SEQ ID NO: 4 and SEQ ID NO: 6, respectively.

The genes provided in the present invention may be introduced into various host cells, and effectively used to produce lycopene and the other carotenoids. The genes may be used alone or in combinations thereof. For example, the crtI gene according to the present invention may be used to produce lycopene by introducing the crtI gene into a microorganism including crtE and crtB genes only. Also, the crtE, crtB and crtI genes according to the present invention may be used to enhance a yield of the lycopene by introducing the crtE, crtB and crtI genes into a microorganism that biosynthesizes carotenoids such as astaxanthin.

Also, the present invention provides a recombinant vector comprising the gene for the biosynthesis of lycopene.

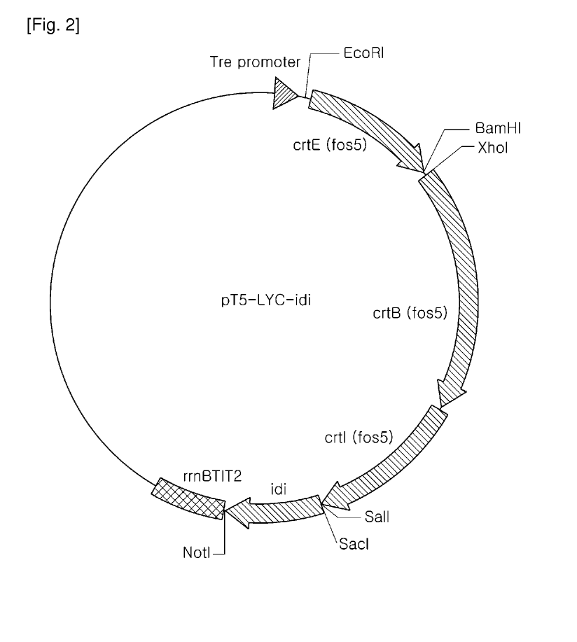

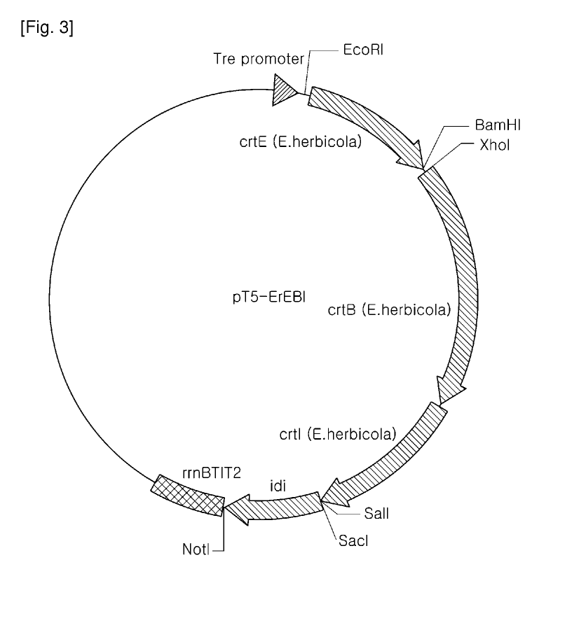

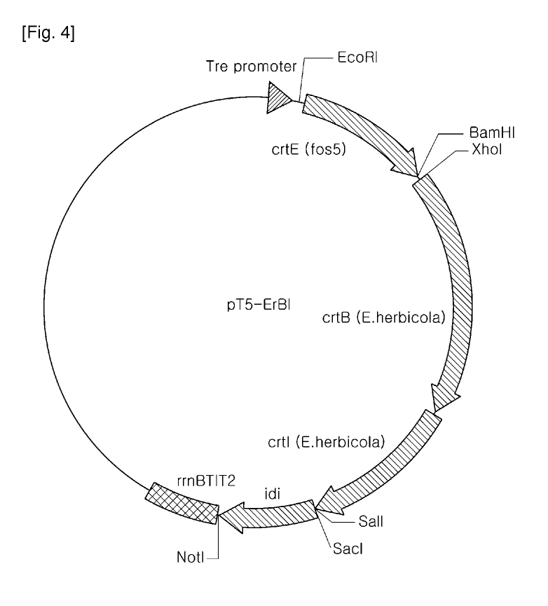

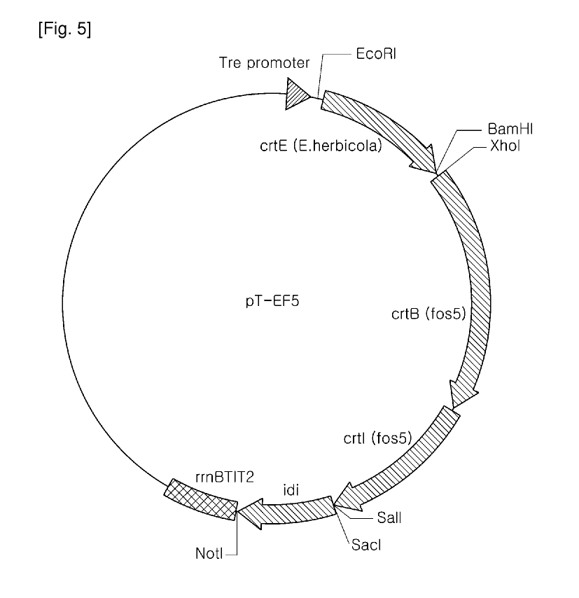

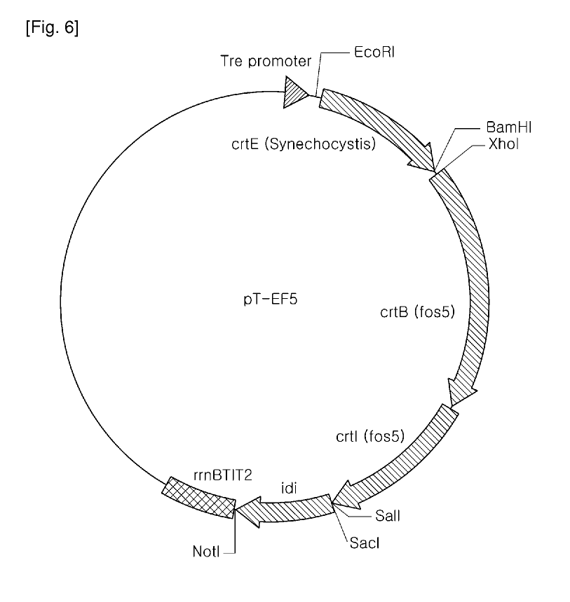

The recombinant vector according to the present invention was constructed by introducing the crtE, crtB and crtI genes into a fundamental vector. All vectors that can be used to clone and express the crt genes may be generally used as the fundamental vector in the present invention, and be varied depending on the host cells. A plasmid pTrc99A was used as the fundamental vector in Examples of the present invention, and a recombinant vector was prepared by introducing crtE, crtB and crtI genes into the fundamental vector and also introducing an idi gene encoding IPP isomerase of E. coli, and was named ‘pT5-LYC-idi (FIG. 2).’ In addition, recombinant vectors were prepared by combining the crt genes of the present invention with the known crt genes, which were named ‘pT5-ErEBI (FIG. 3)’, ‘pT5-ErBI (FIG. 4)’, ‘pT-EF5 (FIG. 5)’ and ‘pT-SF5 (FIG. 6),’ respectively.

In addition to the recombinant vectors, any of recombinant vectors comprising at least one gene selected from the group consisting of the crtE, crtB and crtI genes of the present invention are included in the scope of the present invention.

Also, the present invention provides a transformed strain with the recombinant vector comprising a gene for the biosynthesis of lycopene.

E. coli or yeast may be used as the host that is transformed with the recombinant vector comprising genes for the biosynthesis of lycopene. In Examples of the present invention, transformed E. coli was prepared using the recombinant vector pT5-LYC-idi, pT5-ErEBI, pT5-ErBI, pT-EF5 and pT-SF5.

When an amount of lycopene produced from the transformed strain with the recombinant vector into which the genes are introduced according to the present invention are measured, a yield of the lycopene was 15.3 mg/L (milligram/liter) and a content of the lycopene per cell was 4.2 mg/g (milligram/gram) DCW in E. coli including the combination of the crtE, crtB and crtI genes derived from the metagenome library of seawater. Also, in the E. coli including the combination of the known crt gene and the gene of the present invention, the lycopene was produced at the maximum yield of 22.8 mg/L (milligram/liter) and the maximum content of 5.4 mg/g (milligram/gram) DCW per cell.

As described above, in order to achieve the objects of the present invention, the novel crtE, crtB and crtI genes were obtained from the metagenome library of seawater, and the recombinant vector comprising the gene and the recombinant E. coli transformed with the recombinant vector were also obtained. When the obtained recombinant E. coli strain is subject to the fermentation, the recombinant E. coli strain has a higher lycopene content per cell then the conventional strains in the prior art, which makes it possible to develop an effective production process for lycopene, compared to the prior-art inventions.

Hereinafter, the present invention will be described in more detail in connection with the exemplary embodiments. However, it is understood that the description proposed herein is just a preferable example for the purpose of illustrations only, not intended to limit the scope of the invention.

### MODE FOR THE INVENTION

### Examples

### Example 1

### Cloning Novel Genes (crtE, crtB and crtI) for the Biosynthesis of Lycopene from Metagenome Library of Seawater

In order to obtain crtE, crtB and crtI genes required for the biosynthesis of lycopene, genomic DNA (metagenome) was directly obtained from seawater to construct a metagenome library. On the basis of the fact that lycopene is tinged with red, reddish clones were selected, and sequenced to confirm its identity.

First, microorganisms were collected from a large amount of seawater through the membrane filtration to obtain metagenome DNA from the seawater. Since the most microorganisms have a size of 0.2 to 10 μm (micrometer), various kinds of suspended solids having a size of more than 10 μm (micrometer) were primarily removed by passing a large amount of seawater through a filter having a pore size of 10 μm (micrometer) using a peristaltic pump, and only microorganisms having a size of 0.2 μm (micrometer) or more were then selectively recovered through a filter having a pore size of 0.2 μm (micrometer). The extraction of chromosomal DNA from the recovered microorganisms was carried out according to the method using CTAB (hexadecyltrimethyl ammonium bromide) (Zhou et al., Appl. Environm. Microbiol. 62:316-322, 1996).

A metagenome library was prepared from the metagenome DNA prepared from the resulting microorganism cells using the Copy Control Fosmid library production kit (Epicenter). In this case, the preparation process was carried out according to the manufacturer's manual. The construction of the metagenome library was carried out using Fosmid vector Copy Control pCC1FOS (Epicenter). An insert DNA was ligated into the Copy Control pCC1FOS vector, and the ligated Fosmid clone was then packaged using MaxPlax lambda packaging extracts (Epicenter). In this procedure, more than 10,000 clones were obtained.

The resulting Fosmid clones were stationarily cultivated at a room temperature for 49 hours to observe colors of colonies, and reddish colonies were screened from the cultivated colonies. In order to confirm whether the crt genes are present in these colonies through a PCR method, a pair of primers were synthesized from a crtI C-terminal region (crtIf) and a crtB intermediate region (crtBr) that are derived from Erwinia uredovora, Erwinia herbicola, Flavobacterium sp. strain ATCC21588, Rhodobacter sphaeroides, and Agrobacterium aurantiacum. DNA sequences of the primers were designed, as follows.

| | |
|---|---|
| crtIf: | 5′-GTNGGNGCRGGCACNCAYCC-3′ |
| crtBr: | 5′-TCGCGRGCRATRTTSGTSARRTG-3′ |

The Fosmid DNA extracted from each of the reddish colonies was used as a template, and the synthesized primers were then used with the template to amplify crt genes. That is to say, 100 ng (nanogram) of Fosmid DNA as the template was denatured at 94° C. for 5 minute, and 20 cycles of the PCR amplification were then repeated under the PCR conditions: 94° C., 30 sec.; 50-60° C., 30 sec. and 72° C., 1 min. Then, 15 cycles of the PCR amplification were repeated under the PCR conditions: 94° C., 30 sec.; 50° C., 30 sec. and 72° C., 1 min. As a result, a band having an expected size of 620 bp was obtained from one clone, and inserted into pST-Blue1 vector (Novagen), and its DNA sequence was analyzed. From the DNA sequence analysis, it was confirmed that the cloned DNA sequence has homology to the reported crtB gene.

The resulting fragment of the crtB gene was used as a probe to perform southern blotting thereby to obtain a whole gene cluster for the biosynthesis of lycopene including the crtB gene. The crtB gene fragment used as the probe was attached to DIG dye through the PCR, and the template DNA was digested with each of restriction enzymes BamHI, SalI and EcoRI, and was subject to the southern blotting. First, DNAs digested respectively with the various restriction enzymes were electro-phoresized in 0.9% agarose gel to separate bands of the DNAs by size. Then, the bands of the DNAs were transferred to a nylon membrane (Schleicher & Schuell, Germany) by capillary transfer. The probe was added at 42° C. to a stock solution (5×SSC, 0.1% N-Lauroylsarcosine, 0.02% SDS, 5% Blocking regent, 50% Formamide) including 50% formamide, and the hybridization was then carried out for 6 hours or more. The nylon membrane reacts with an antibody against DIG bound to alkaline phosphatase according to the manufacturer's manual (Boehringer-Mannheim, Germany), and NBT and X-phosphate were added as substrates to perform a color reaction.

As a result of the southern blotting a band with about 4 kb among the Eco RI-restricted DNAs showing a signal was introduced into a pBluescript II KS (+) vector (Stratagene) to sequence a DNA fragment. From the sequencing result, it was revealed that the band has a cluster including crtE, crtB and crtI genes having the total 3.2 kb. As described above, the crtE, crtB and crtI genes were cloned from the metagenome library of seawater. In this case, the crtE, crtB and crtI genes had different DNA sequences from the known genes.

The following primers are designed on the basis of the DNA sequence of the crt gene cluster, and used in the PCR reaction. Then, the about 3.2-kb DNA fragment including three crt genes was cloned between XhoI and XbaI restriction sites in the pBluescriptII KS (+) vector, and named ‘pBF5-crt’.

F5crt-F:
5′-GTCTCGAGAGGAGGTAATAAATATGATAAGCCCTATATCCACT
GCTGAT-3′
F5crt-R1:
5′-GATTCTAGATCTAAACCCTCACTGCC-3′

### Example 2

Preparation of recombinant vector including genes for the biosynthesis of lycopene derived from metagenome library of seawater

The crtE, crtB, crtI genes cloned in Example 1 were inserted into an expression vector pTrc99A (Amannm E. et al., (1998) Gene, 69:301-305).

First, a pair of the following primers were synthesized to insert the crtE gene into a pTrc99A vector.

f5E-f:
5′-TGGAATTCTACATCAGGAGGTAATAAATATGATAAGCCCTATA
TCCAC-3′
f5E-r:
5′-TAGGATCCCTCGAGATGCATTATCATGGGAGCTTCGCTCGGAG
C-3′

The vector pBF5-crt prepared in Example 1 was used a template, and amplified using the primers to obtain a DNA fragment including a crtE gene with about 0.85 kb. The resulting DNA fragment was purified using a Qiagen PCR purification kit (Qiagen), digested with restriction enzymes EcoRI and BamHI and introduced into a pTrc99A vector that was digested with the same restriction enxaymes, which was named pT-f5crtE. Next, two pairs of the following primers were synthesized to introduce the crtB and crtI genes into the vector pT-f5crtE.

f5I-f:
5′-ATCTCGAGAGGAGGTAATAAATATGCAAACAGTTGTTATTG
GTG-3′
f5I-r:
5′-CTCCTCTGCAGTTATCATGGCTGCTCCGCAGTCACCAC-3′
f5B-f:
5′-CCATGATAACTGCAGAGGAGGTAATAAATATGAAGATAGCG
CTGGACCGG-3′
f5B-r:
5′-AGGTCGACGCGGCCGCGAGCTCTTATCGTAAACCCTCACTG
CCAAC-3′

First, the vector pBF5-crt was used a template, and amplified using the primers f5I-f and f5I-r to obtain a DNA fragment including a crtI gene with about 1.5 kb, and the resulting DNA fragment was purified using a Qiagen PCR purification kit. Then, the vector pBF5-crt was used a template, and amplified using the primers f5B-f and f5B-r to obtain a DNA fragment including a crtB gene with about 0.9 kb, and the resulting DNA fragment was purified using a Qiagen PCR purification kit. The two DNA fragments obtained thus were mixed with each other, and amplified in the PCR reaction using the primers f5I-f and f5B-r to obtain the final DNA fragment including the crtB and crtI genes with about 2.4 kb. The resulting DNA fragment was purifies using a Qiagen PCR purification kit, digested with restriction enzymes XhoI and SalI, and introduced into a vector pT-f5crtE that is digested with the same restriction enzymes, which was named pT-f5EBI. Then, a pair of the following primers idi-f and idi-r were synthesized to introduce an idi gene encoding IPP isomerase of E. coli into the vector pT-f5EBI.

idi-f:
5′-TAAHAHCTCTAATAAATATHCAAACHHAACACHTCAT-3′
idi-r:
5′-CGACGCGGCCGCGCTTATTTAAGCTGGGTAAATGC-3′

Chromosomal DNA of E. coli MG1655 was subject to PCR using a pair of the primers to obtain a DNA fragment containing an idi gene with about 0.6 kb, and the resulting DNA fragment was purified using a Qiagen PCR purification kit. The purified DNA fragment was digested with restriction enzymes SacI and NotI, and introduced into the vector pT-f5EBI that is digested with the same restriction enzymes, which was named pT5-LYC-idi (FIG. 2).

### Example 3

### Production of Lycopene in Recombined E. coli

It was confirmed whether the biosynthesis of lycopene proceeds in an E. coli strain transformed with the vector pT5-LYC-idi prepared in Example 2.

First, an E. coli MG1655 was transformed with the vector pT5-LYC-idi. Each of single colonies of the transformed E. coli was inoculated in 5 mL (milliliter) of 2YT medium (16 g/L trypton, 10 g/L yeast extract and 5 g/L NaCl) supplemented with 100 μg/mL (microgram/milliliter) of ampicillin and 50 μg/mL (microgram/milliliter) of chloramphenicol, incubated at 37° C. for 8 hours while shaking. 600 μl (microliter) of the resulting culture broth was inoculated in 30 ml (milliliter) of 2YT medium supplemented with 1% glycerol and 100 μg/mL (microgram/milliliter) of ampicillin, and incubated at 30° C. for 48 hours.

When the cell culture was completed, a suitable amount of the culture broth was taken to confirm the productivity of lycopene by calculating dry cell weight (gDCW/L), yield (mg Lycopene/L, hereinafter, referred to as ‘mg/L’), content (mg Lycopene/gDCW, hereinafter, referred to as ‘mg/gDCW’) of the lycopene.

First, in order to obtain dry cell weight of lycopene, 5 mL (milliliter) of the strain culture broth was taken and put into a 50 mL (milliliter) centrifuge tube, centrifuged (8,000 rpm, 10 min.) to remove a supernatant and recover a cell pallet. The recovered cell pallet was added to 20 mL (milliliter) of sterile distilled water, and suspended, and centrifuged to completely remove culture broth components and recover a cell pallet. The recovered cell pallet was added to 5 mL (milliliter) of sterile distilled water, completely suspended, and then put on an aluminum weighing dish that was previously weighed by mg (milligram) unit. In this case, the centrifuge tube was washed with sterile distilled water, and the washed solution was also added to a weighing dish. The weighing dish was dried at 105° C. for 12 hours or more in a dry oven, and cooled to measure the weight of the weighing dish by mg (milligram) unit. The dry cell weight (gDCW/L) was calculated using the following Equation 1.

Equation 1

Dry cell weight (gDCW/L)={dish weight after drying (mg)−dish weight (mg)}/5

In order to determine a yield of the lycopene, the culture broth were centrifuged at an amount of 100 μl (microliter) to obtain cell pellets, and each of the cell pellets was suspended in 400 μl (microliter) of acetone, and kept at 55° C. for 15 minutes. 600 μl (microliter) of acetone was added again to the resulting suspension, and the lycopene was extracted by keeping the suspension at 55° C. for 15 minutes. The resulting extract was centrifuged at a rotary speed of 14,000 rpm for 10 minutes to separate a supernatant. Then, the resulting separated supernatant was measured for absorbance at a wavelength of 474.5 nm (nanometer) using a spectrophotometer. Then, the measured values were subject to an equation obtained through the calibration curve, and an amount of the lycopene was determined by calculating a dilution rate. In this case, in order to plot a calibration curve, the standard lycopene (Sigma) was purchased, dissolved in acetone, and diluted with different concentrations. Then, the diluted standard lycopenes were measured for absorbance at 474.5 nm (nanometer) wavelength using a spectrophotometer, and the resulting absorbance values were used to plot the standard calibration curve.

The content (mg/gDCW) of lycopene was calculated from the following Equation 2 using the dry cell weight (gDCW/L) and yield (mg/L) of the lycopene.

Equation 2

Content (mg/gDCW)=yield (mg/L)/dry cell weight (gDCW/L)

A level of the produced lycopene determined from the equation is listed in the following Table 1.

TABLE 1

| Dry cell weight (gDCW/L) | Yield (mg/L) | Content (mg/gDCW) |
|---|---|---|
| 3.56 | 15.3 | 4.2 |

### Example 4

### Evaluation of Lycopene Productivity in Transformed E. Coli with Recombinant Vector Including Erwinia herbicola-Derived crtE, crtB and crtI Genes

A vector pT5-ErEBI (FIG. 3) was prepared using the obtained Erwinia herbicola-derived crtE, crtB and crtI genes, and introduced into E. coli to obtain a transformed E. coli strain. Then, the transformed E. coli strain was evaluated for productivity of lycopene in the same manner as in Example 3. After the culture for 48 hours, the productivity of the obtained lycopene was listed in the following Table 2.

TABLE 2

| Dry cell weight (gDCW/L) | Yield (mg/L) | Content (mg/gDCW) |
|---|---|---|
| 3.7 | 12.7 | 3.5 |

### Example 5

### Evaluation of Lycopene Productivity in Transformed E. coli with Recombinant Vector Including Combination of Novel crtE Gene and Erwinia herbicola-Derived crtB and crtI Genes

A recombinant vector pT5-ErBI (FIG. 4) was prepared by substituting the crtB and crtI genes in the vector pT5-LYC-idi obtained in Example 2 with corresponding known Erwinia herbicola-derived genes.

The transformed E. coli with the recombinant vector pT5-ErBI was obtained and evaluated for productivity of the novel crtE gene in the same manner as in Example 3. After the culture for 48 hours, the productivity of the obtained lycopene was listed in the following Table 3.

TABLE 3

| Dry cell weight (gDCW/L) | Yield (mg/L) | Content (mg/gDCW) |
|---|---|---|
| 4.9 | 10.6 | 2.2 |

### Example 6

### Evaluation of Lycopene Productivity in Transformed E. coli with Recombinant Vector Including Combination of Erwinia herbicola-Derived crtE Gene and Novel crtB and crtI Genes

A recombinant vector pT-EF5 (FIG. 5) was prepared by substituting the crtE gene in the vector pT5-LYC-idi obtained in Example 2 with a corresponding Erwinia herbicola-derived gene.

The transformed E. coli with the recombinant vector pT-EF5 was obtained and evaluated for productivity of the novel crtB gene and the novel crtI gene in the same manner as in Example 3. After the culture for 48 hours, the productivity of the obtained lycopene was listed in the following Table 4.

TABLE 4

| Dry cell weight (gDCW/L) | Yield (mg/L) | content (mg/gDCW) |
|---|---|---|
| 4.2 | 22.8 | 5.4 |

### Example 7

### Evaluation of Lycopene Productivity in Transformed E. coli with Recombinant Vector Including Combination of Synechocystis Sp.PCC6803-Derived crtE Gene and Novel crtB and crtI Genes

A recombinant vector pT-SF5 (FIG. 6) was prepared by substituting the crtE gene in the vector pT5-LYC-idi obtained in Example 2 with a corresponding Synechocystis sp. PCC6803-derived gene.

The transformed E. coli with the recombinant vector pT-SF5 was evaluated for productivity of the lycopene in the same manner as in Example 3. Then, the productivity of the obtained lycopene was listed in the following Table 5.

TABLE 5

| Dry cell weight (gDCW/L) | Yield (mg/L) | Content (mg/gDCW) |
|---|---|---|
| 4.1 | 19.5 | 4.8 |

### Sequence Listing

SEQ ID NO: 1 is a DNA sequence (867 bp) of crtE gene derived from metagenome in the seawater.

SEQ ID NO: 2 is an amino acid sequence (288 amino acids) of geranylgeranyl pyrophosphate synthase encoded by crtE gene.

SEQ ID NO: 3 is a DNA sequence (909 bp) of crtB gene derived from metagenome in the seawater.

SEQ ID NO: 4 is an amino acid sequence (302 amino acids) of phytoene synthase encoded by crtB gene.

SEQ ID NO: 5 is a DNA sequence (1,485 bp) of crtI gene derived from metagenome in the seawater.

SEQ ID NO: 6 is an amino acid sequence (494 amino acids) of phytoene desaturase encoded by crtI gene.

SEQ ID NO: 7 is a DNA sequence of crtE gene in Synechocystis sp. PCC 6803.

1. A crtE gene encoding geranylgeranyl pyrophosphate synthase and having a DNA sequence set forth in SEQ ID NO: 1.

2. A crtB gene encoding phytoene synthase and having a DNA sequence set forth in SEQ ID NO: 3

3. A crtI gene encoding phytoene desaturase and having a DNA sequence set forth in SEQ ID NO: 5.

4. A recombinant vector comprising at least one gene selected from the group consisting of the crtE gene set forth in SEQ ID NO: 1, the crtB gene set forth in SEQ ID NO: 3, and the crtI gene set forth in SEQ ID NO: 5.

5. The recombinant vector of claim 4, comprising the crtE gene set forth in SEQ ID NO: 1, the crtB gene set forth in SEQ ID NO: 3, and the crtI gene set forth in SEQ ID NO: 5.

6. The recombinant vector of claim 4, comprising the crtB gene set forth in SEQ ID NO: 3, and the crtI gene set forth in SEQ ID NO: 5, and further comprising crtE gene set forth in SEQ ID NO: 7.

7. The recombinant vector of claim 4, comprising the crtB gene set forth in SEQ ID NO: 3, and the crtI gene set forth in SEQ ID NO: 5, and further comprising crtE gene derived from Erwinia herbicola.

8. A transformed microorganism with recombinant vector defined in claim 4.

9. The transformed microorganism of claim 8, comprising E. coli.

10. A transformed microorganism with recombinant vector defined in claim 5.

11. A transformed microorganism with recombinant vector defined in claim 6.

12. A transformed microorganism with recombinant vector defined in claim 7.


SEQUENCE LISTING (19 sequences)

SEQ ID NO: 1 - DNA, 867 bp, Unknown
  Description of Unknown crtE polynucleotide
atgataagccctatatccactgctgatgtggcctttgagcgcctcgttgacagctgtgaacgatcgttgaaagagtgtatagccgcgagctgtccagcccttcatcaagcttggcagcatcagttcgcagcgcgaggcaagcgtttacgtatgcacctagccttagaaagtagtctggcgctagggttgaccgaccatcaatgccacaccattgcggtggcatgcgaattagtccaccaggcctcattgattcacgatgatgtgcttgatgcggatacccaccgaaatggcaaagcaacggtttggcaccagtatggagctgccacagcaatttgtctgggtgacagtttattagttgaggcaatgctgcaaatagcgttgttggaaaatttaccgagcgccgttcggcagcagcttgtgcaattatttaaagatgccatacaagccgccgctgagggccaaattgacgattgtaatagcgacaaaatagccaactatagccatgccgattattgcactgcagtgcgcaaaaaatcaggcgcgctgttcggcttaccggtgttggcggctatgttaatgagtcaacagcatgcaattactatcggggtagccaaccgagcctatgctgaatttggtattgcctatcagttactcgatgacctgcatgaccgtgacgttgatcagcagggtcggatgaacggttattgggtattaagtcgggattatccgaccggggtagaagcagcactctttgctgcggttgagcagcatctcggcgaggccgagcgactgatcgcatcattgccatcgagcttgcaccccagcttttatgtggtgcatgactcgctccgagcgaagctcccatga

SEQ ID NO: 2 - protein, 288 aa, Unknown
  Description of Unknown Geranylgeranyl pyrophosphate synthase polypeptide
MISPISTADVAFERLVDSCERSLKECIAASCPALHQAWQHQFAARGKRLRMHLALESSLALGLTDHQCHTIAVACELVHQASLIHDDVLDADTHRNGKATVWHQYGAATAICLGDSLLVEAMLQIALLENLPSAVRQQLVQLFKDAIQAAAEGQIDDCNSDKIANYSYADYCTAVRKKSGALFGLPVLAAMLMSQQHAITIGVANRAYAEFGIAYQLLDDLHDRDVDQQGRMNGYWVLSRDYPTGVEAALFAAVEQHLGEAERLIASLPSSLHPSFYVVHDSLRAKLP

SEQ ID NO: 3 - DNA, 909 bp, Unknown
  Description of Unknown crtB polynucleotide
atgaagatagcgctggaccggcctgagcatgctgccattatgcagcagcatggcaagtcattttatttggctggtagctttctcggtcgtgatgcctggcagcgtgcgtcagcgctttatgcttttttacgccatatcgacgaccaaattgatgaagctgaaacatctgccgtagcagcgcaacgactggcacagattcgtcagcagctgttctcaagcgcaatcatgaccgacgcagatgagcagagcttaagcattgagcaaagcaccctggagcaatttttgcgtggcatggcttatgacattggtcacgttgctattgctgatcaggctgagttagaagactactgctattgtgtcgccggcaccgtcggtgaaatgatgtgtcaggccttgcgctgtgatgacccgcgcgcaattggtcatgctattgatttgggtgtcgctatgcaaatgaccaatattgcccgcgatgttcatgccgatagcgccttagggcgccgttatttacccgccacctgggttggtgatctcagtgctgagagcattaccacggcaacaccagctatctcggcacagatagccgcggcaattatgcggctgattgcgttatctgagcagcgttatcaatcagcgtatgcgggtatcgcactgttgccgttgcgctcgcgcttggcaattttggcggcaagtcacctttatgccggtattggtcgcgccattgcggcggagcatgcgcaatcatggcagcaacggaaggtgttgtcagggtcgcgtaaggcggcaattactgccgccgcagtggcggaatttgcgactcgaccgcgactatggcgttattacgcgcagcctagcttcggtaagccggccgagcggatcgctgcgtctgttggcagtgagggtttatga

SEQ ID NO: 4 - protein, 302 aa, Unknown
  Description of Unknown Phytoene synthase polypeptide
MKIALDRPEHAAIMQQHGKSFYLAGSFLGRDAWQRASALYAFLRHIDDQIDEAETSAVAAQRLAQIRQQLFSSAIMTDADEQSLSIEQSTLEQFLRGMAYDIGHVAIADQAELEDYCYCVAGTVGEMMCQALRCDDPRAIGHAIDLGVAMQMTNIARDVHADSALGRRYLPATWVGDLSAESITTATPAISAQIAAAIMRLIALSEQRYQSAYAGIALLPLRSRLAILAASHLYAGIGRAIAAEHAQSWQQRKVLSGSRKAAITAAAVAEFATRPRLWRYYAQPSFGKPAERIAASVGSEGL

SEQ ID NO: 5 - DNA, 1485 bp, Unknown
  Description of Unknown crtI polynucleotide
atgcaaacagttgttattggtggaggcttaggtggtatcgcagcggcgttgcgagcccgtgcaaaaggccatcaagtcaccctaatagaaaaaaatcagcagttaggtggccgtgcgcaagtatttgaacgtgagggttttcgttttgatgccggccccaccgtgattactgcaccattcttgtttgatgagctatttgaattatttggcaaaaaacgccaagactatgtcgagtttattccgctcaatccgtggtaccaattttactacagtgacgacaagtcgcgcttcaactatggtggaagtgtcgatgacaccttgcaagaaattgctaaaattgagccaagtgaccaggccaattatctgcgtttaatcgagcatagcaaaaagatctacaaaatcggctttgagcaactcgccgatcagccgtttcacaagctttccaccatgttaaagcaaattccccatttgggccggctgcgcgctgaccgcacggtttggaatatggttagtcgctatcttaaaaatgacaaactacgccaagctttttctattcagtcattgctagtaggtggtaacccatttgataccaccagtatttatggactgattcattatttagagcgggaatatggcattcatttcgccatgggcggcaccggtgccattattgatgcattacacaagctgatgctcgaagagggtatcgaggtgcgcacgaactgctgtgtcaccgactttcatagcagcccgagccgcattgagagcgcagtgattaatcagcacgaggtgctatctgctgactactttatttttaatggcgacccactgtatttgtataaacacctgttacctgaaagttctgctaatttgcaattacggttgaaggttgatcacagtaaacgctcaatgggtctatatgtgctgttttttggcaccaccaaacaatatccagaggttgagcatcacactatttggctgggcaagcgttatcagcaattattagcagaaatttttgccgaaaaatcattacccgatgatttttcactttatgtacatagaccaactgcttcggatccatcctttgcgccggctggttgcgacagcttttatgtgttagctccggtgcccaatctgcgggcagatatagattggcaggttgaggaacccaagttgcgacaacggatcatcgacgcgctagcagataccttattgccgggcttacatgactgtattaccgctgagtttgcgatgaccccagaacagtttaaaagcgattatttgagtgtcgatggcgctggcttttccattgcacccaaatttactcagtcggcgtggttccgttttcataatctgtcggaaaaatatagcaacttattactcgctggtgccggaacgcacccaggtgctggcatgccgggcgtactctgttcggcaaaagtcattgaaaaactgctccctgtggtgactgcggagcagccatga

SEQ ID NO: 6 - protein, 494 aa, Unknown
  Description of Unknown Phytoene desaturase polypeptide
MQTVVIGGGLGGIAAALRARAKGHQVTLIEKNQQLGGRAQVFEREGFRFDAGPTVITAPFLFDELFELFGKKRQDYVEFIPLNPWYQFYYSDDKSRFNYGGSVDDTLQEIAKIEPSDQANYLRLIEHSKKIYKIGFEQLADQPFHKLSTMLKQIPHLGRLRADRTVWNMVSRYLKNDKLRQAFSIQSLLVGGNPFDTTSIYGLIHYLEREYGIHFAMGGTGAIIDALHKLMLEEGIEVRTNCCVTDFHSSPSRIESAVINQHEVLSADYFIFNGDPLYLYKHLLPESSANLQLRLKVDHSKRSMGLYVLFFGTTKQYPEVEHHTIWLGKRYQQLLAEIFAEKSLPDDFSLYVHRPTASDPSFAPAGCDSFYVLAPVPNLRADIDWQVEEPKLRQRIIDALADTLLPGLHDCITAEFAMTPEQFKSDYLSVDGAGFSIAPKFTQSAWFRFHNLSEKYSNLLLAGAGTHPGAGMPGVLCSAKVIEKLLPVVTAEQP

SEQ ID NO: 7 - DNA, 909 bp, Synechocystis sp.
atggttgcccaacaaacacgaaccgactttgatttagcccaatacttacaagttaaaaaaggtgtggtcgaggcagccctggatagttccctggcgatcgcccggccggaaaagatttacgaagccatgcgttattctctgttggcggggggcaaacgattgcgaccgattttatgcattacggcctgcgaactgtgtggcggtgatgaagccctggccttgcccacggcctgtgccctggaaatgatccacaccatgtccctcatccatgatgatttgccctccatggataatgacgatttccgccggggtaaacccactaaccacaaagtgtacggggaagacattgccattttggccggggatggactgctagcctatgcgtttgagtatgtagttacccacaccccccaggctgatccccaagctttactccaagttattgcccgtttgggtcgcacggtgggggccgccggtttagtggggggacaagttctagacctggaatcggaggggcgcactgacatcaccccggaaaccctaacttttatccatacccataaaaccggggcattgctggaagcttccgtgctcacaggcgcaattttggccggggccactggggaacaacaacagagactggcccgctatgcccagaatattggcttagcttttcaagtggtggatgacatcctcgacatcaccgccacccaggaagagttgggtaaaaccgctggtaaagatgtcaaagcccaaaaagccacctatcccagtctcctcggtttggaagcttcccgggcccaggcccaaagtttgattgaccaggccattgtcgccctggaaccctttggcccctccgccgagcccctccaggcgatcgccgaatatattgttgccagaaaatattga

SEQ ID NO: 8 - DNA, 20 bp, Artificial Sequence
  Description of Artificial Sequence Synthetic primer
gtnggngcrggcacncaycc

SEQ ID NO: 9 - DNA, 23 bp, Artificial Sequence
  Description of Artificial Sequence Synthetic primer
tcgcgrgcratrttsgtsarrtg

SEQ ID NO: 10 - DNA, 49 bp, Artificial Sequence
  Description of Artificial Sequence Synthetic primer
gtctcgagaggaggtaataaatatgataagccctatatccactgctgat

SEQ ID NO: 11 - DNA, 26 bp, Artificial Sequence
  Description of Artificial Sequence Synthetic primer
gattctagatctaaaccctcactgcc

SEQ ID NO: 12 - DNA, 48 bp, Artificial Sequence
  Description of Artificial Sequence Synthetic primer
tggaattctacatcaggaggtaataaatatgataagccctatatccac

SEQ ID NO: 13 - DNA, 44 bp, Artificial Sequence
  Description of Artificial Sequence Synthetic primer
taggatccctcgagatgcattatcatgggagcttcgctcggagc

SEQ ID NO: 14 - DNA, 44 bp, Artificial Sequence
  Description of Artificial Sequence Synthetic primer
atctcgagaggaggtaataaatatgcaaacagttgttattggtg

SEQ ID NO: 15 - DNA, 38 bp, Artificial Sequence
  Description of Artificial Sequence Synthetic primer
ctcctctgcagttatcatggctgctccgcagtcaccac

SEQ ID NO: 16 - DNA, 50 bp, Artificial Sequence
  Description of Artificial Sequence Synthetic primer
ccatgataactgcagaggaggtaataaatatgaagatagcgctggaccgg

SEQ ID NO: 17 - DNA, 46 bp, Artificial Sequence
  Description of Artificial Sequence Synthetic primer
aggtcgacgcggccgcgagctcttatcgtaaaccctcactgccaac

SEQ ID NO: 18 - DNA, 37 bp, Artificial Sequence
  Description of Artificial Sequence Synthetic primer
taahahctctaataaatathcaaachhaacachtcat

SEQ ID NO: 19 - DNA, 35 bp, Artificial Sequence
  Description of Artificial Sequence Synthetic primer
cgacgcggccgcgcttatttaagctgggtaaatgc